(19) United States
(12) Patent Application Publication
(10) Pub. No.: US 2004/0132015 A1
(43) Pub. Date: Jul. 8, 2004

(54) DNA chip using codon scanning algorithm

(76) Inventors: Sang Yup Lee, Taejon (KR); Jong Gil Choi, Taejon (KR)

Correspondence Address: KNOBBE MARTENS OLSON & BEAR LLP, 2040 MAIN STREET, FOURTEENTH FLOOR, IRVINE, CA, 92614, US

(21) Appl. No.: 09/963,314

(22) Filed: Sep. 25, 2001

(30) Foreign Application Priority Data
Sep. 29, 2000 (KR) 10-2000-57208

(51) Int. Cl. C12Q 1/68; C12P 19/34

(52) U.S. Cl. 435/6; 435/91.2

(57) ABSTRACT

The present invention relates to a process for preparing oligonucleotide probes which are designed to detect mutations in the entire interrogated codon regions determined by codon scanning algorithm, a process for preparing DNA chip using the probes prepared by the said process, a DNA chip prepared by the said process, and a method for detecting mutations using the said DNA chip. The process for preparing probes of the invention comprises the steps of: selecting mutated codon to be interrogated; and, preparing the probes such that the interrogated mutated codon is located at the center-most position of the oligonucleotide probe consisting of 7 nucleotides or more, rest of sequences are remained same as those of normal individuals and amine group is linked to 3′ terminus of the probe, and the process for preparing DNA chip comprises a step of immobilizing the said probes on solid surface. By using the DNA chip of the invention, errors made in interpretation of results due to base pair mismatches found with DNA probes designed by conventional algorithms can be avoided, homozygous mutations can be discerned from heterozygous mutations, mutations causing various genetic diseases can be detected and identified in a rapid and accurate manner, and DNA chip using codon scanning algorithm can be applied for the diagnosis of all kinds of genetic mutation-associated diseases and the identification of mutations such as SNP.

### FIELD OF THE INVENTION

The present invention relates to a DNA chip, more specifically, to a process for preparing oligonucleotide probes which are designed to detect mutations in the entire interrogated codon regions determined by codon scanning algorithm of the invention, a process for preparing DNA chip using the probes prepared by the said process, a DNA chip prepared by the said process, and a method for detecting genetic mutations using the said DNA chip.

### BACKGROUND OF THE INVENTION

As the Human Genome Project is approaching completion, genes, the basis of the life phenomena, are attracting interests. Among approximately 100,000 human genes estimated, approximately 10,000 genes have been identified and most of them are directly related with genetic diseases. Thus, increasing is attention on the researches on genetic diseases caused by malfunctioning of the proteins expressed from the mutated disease-related genes and diagnostic procedures of the diseases. If the genetic diseases identified by now could be diagnosed early enough, onset of the disease could be avoided, however, lack of diagnostic tool and information on the diseases limit the early diagnosis of the disease. In view of above situation, there is a need to develop techniques to solve said problems and DNA chip technology newly emerged recently is presented as one of the solutions.

For example, DNA chip technology in which large number of DNA probes are immobilized on the surface of solid supports such as glass, is being spotlighted for the advantage of meeting the demands for rapid and large scale analysis under various conditions which was impossible by conventional technologies (see: Shena, M., et al, Science, 270:467-470, 1995). Application area of DNA chip is divided into two main fields, analysis of expression of the gene and analysis of mutations such as SNP (single nucleotide polymorphism) (see: Halushka, M., et al, Nature Genetics, 22:239-247, 1999). Currently, the most popular DNA chip used for analysis of SNP or mutations is the chip called GeneChip™ manufactured by Affymetrix, Calif., USA (see: www.affymetrix.com). GeneChip™ is manufactured employing photolithographic process which is used in the production of semiconductors, and solid phase synthesis technique, which is employed to synthesize DNA in situ on the solid supports, and these techniques employed for manufacturing of DNA chip carrying over 100,000 DNA probes have an advantage to prepare multiplicity of DNA fragments in a uniform amount in a simultaneous manner. However, such photolithographic technique needs the use of photomask which demands intensive labor and high cost to manufacture. Furthermore, this technique has following disadvantages that enormous equipments are needed through entire manufacturing process of DNA chip, keeping the manufacturing cost of DNA chip high, it is impossible to prepare a variety of probes needed only, and additional probes cannot be supplemented into the chip already prepared. For these reasons, photolithography is not a proper choice as a technique for manufacturing DNA chip to screen mutations of interest selectively.

On the other hand, the spotting technique which is used for immobilizing the pre-synthesized probe of interest onto the solid support, has been employed in manufacturing DNA chip. In the spotting technique, the method of immobilizing probes varies depending on the type of probe used: for the cDNA chip which employs PCR (polymerase chain reaction) products as probes, the probes can be immobilized on the support by chemical reaction between thymine group of PCR product and amine group of polylysine on the solid surface (see: Southern E., et al, Nature Genetics, 21:5-9, 1999), however, for the DNA chip which employs DNA fragment as a probe, the above chemical reaction is not a feasible method to immobilize probes due to the short length of DNA fragments (20 nt or so). Because of the problems described above, only DNA chips manufactured by photolithograhy in which probes are synthesized in situ on the solid support have been commercialized by now rather than DNA chips prepared by spotting DNA fragments onto nylon membrane or glass plated covered with polymer gel. Therefore, urgently required in the art is to develop DNA chip which can be applied to diagnosis of mutations without using expensive photolithograhic technique to immobilize DNA probes.

Until now, two methods have been reported for design of DNA probes required for detecting mutations (see: Hacia, J. D., et al., Nature Genetics, 21:42-47, 1999), one is the gain of signal algorithm in which complementary probes are prepared to all possible mutations and the other is the loss of signal algorithm in which loss of complementary bindings are detected by scanning of target DNA segment one nucleotide at a time with oligonucleotide probes of certain length. These algorithms are not used for detecting selected mutations but used for screening of mutations by analysing entire nucleotide sequence by using a large number of probes manufactured by photolithography.

The gain of signal algorithm is that the signal would be found only for the perfectly matched probe to the sequence of area of interrogated, and DNA probes with all possible nucleotide (A, G, T and C) inserted are prepared for the every position of mutation. Therefore, all possible number of oligonucleotide probes complementary to the target DNA segment with sequence variations of substitution, insertion and deletion at a particular location would be prepared. Now, although the nucleotide sequence analysis using probes designed in this way shows greater than 90% accuracy, there are problems that it is difficult to exclude base pair mismatch in case of insertion mutation, and it is practically impossible to detect and analyze mutation without optimizing hybridization and washing condition due to the mismatch occurred inevitably during hybridization reaction.

The loss of signal algorithm is that the loss of hybridization signal due to the base pair mismatch between DNA of interrogation and the probe is detected, and has characteristics of overlaps between probes which are prepared in a uniform length with one nucleotide shift at a time. When the loss of signal algorithm is used, heterozygous mutations would show 50% loss of signal intensity and homozygous mutation would show 100% loss of signal intensity relative to the wild type target DNA. With the use of loss of signal algorithm, loss of complementary binding would be found in many overlapped probes, allowing detection of mutations with fidelity. However, the loss of signal analysis has a disadvantage that the mutation cannot be discerned and the identity of mutation must be established by subsequent sequencing of the region surrounding the loss of signature. Also, another limitation reside in this method that, to distinguish homozygous mutations from heterozygous mutations, the quantity of immobilized probes should be kept even, which is attainable only with expensive photolithography.

Therefore, to solve the problems mentioned above, there is a continuing need to develop a DNA chip which can identify mutations in an accurate and economical way.

### SUMMARY OF THE INVENTION

The present inventors have made an effort to develop a DNA chip which can identify mutations in an accurate and economical way, thus, a set of three consecutive nucleotides containing a mutation to be identified was selected as an interrogated mutated codon, the probes were prepared based on the codon scanning algorithm by which interrogated codons are selected, and the DNA chip was prepared employing spotting technique and amine-aldehyde reaction with which the said probes can be immobilized at the intended position, and they have found that the mutations can be identified in an accurate and economical way employing the DNA chip prepared by the said process.

The first object of the present invention is, therefore, to provide a process for preparing probes using codon scanning algorithm which can identify genetic mutations.

The second object of the invention is to provide a process for preparing DNA chip using the probes prepared by the said process.

The third object of the invention is to provide DNA chip prepared by the said process.

The fourth object of the invention is to provide a method for detecting genetic mutations employing the said DNA chip.

### BRIEF DESCRIPTION OF THE DRAWINGS

The above and the other objects and features of the present invention will become apparent from the following descriptions given in conjunction with the accompanying drawings, in which:

FIG. 1 is a schematic representation of the DNA chip immobilized with 12 probes each for the 14 regions to be interrogated.

FIG. 2a is a photograph showing the DNA chip before treatments of binding and washing.

FIG. 2b is a photograph showing the DNA chip after treatments of binding and washing.

FIG. 3a is a graph showing the analysis of mutation of normal individual.

FIG. 3b is a graph showing the analysis of mutation of Wilson disease patients.

FIG. 4a is a graph showing the analysis of mutation frequently found in Wilson disease patients in Korean population using DNA chip of the invention.

FIG. 4b is a graph showing the analysis of mutation frequently found in Wilson disease patients in western population and in phosphate domain using DNA chip of the invention.

FIG. 4c is a graph showing the analysis of mutation found in ATP binding domain using DNA chip of the invention.

FIG. 4d is a graph showing the analysis of mutation found in hinge domain using DNA chip of the invention.

FIG. 5 is a graph showing the analysis of Arg778Leu mutation in the patient DNA.

FIG. 6 is a graph showing the analysis of multiplex PCR amplified DNA from normal individual.

FIG. 7a is a graph showing the analysis of Ala874Val mutation in the DNA from a patient using the DNA chip of the invention.

FIG. 7b is a graph showing the analysis of Leu1083Phe mutation in the DNA from a patient using the DNA chip of the invention.

### DETAILED DESCRIPTION OF THE INVENTION

The present inventors manufactured DNA chip by immobilizing the probes designed and prepared using codon scanning algorithm on the solid surface employing a spotting technique. The codon scanning algorithm is an algorithm which has taken advantages of gain of signal algorithm and loss of signal algorithm to make up the problems found in probes designed by each algorithm. The process for preparing probes using the said algorithm comprises steps of: selection of mutated codon to be interrogated; and preparing the probes such that the interrogated mutated codon is located at the center-most position of the oligonucleotide probe consisting of 7 nucleotides or more, rest of sequences are remained same as those of normal individuals and amine group is linked to 3′ terminus of the probe.

The process for preparing the probes of the present invention is further illustrated in the following steps.

Step 1: Selection of Mutated Codon to be Interrogated

Mutated codons found in the patients with a particular genetic disease are selected as interrogated mutated codons: in selecting interrogated codons found in the patients with a particular genetic disease, the mutated codon containing a changed nucleotide sequence is selected as a mutated codon to be interrogated if the mutation occurred in the gene, and the codon coding for the mutated amino acid is selected as a mutated codon to be interrogated if the amino acid was changed, resulting in N mutated codons to be interrogated for one particular genetic disease, wherein N is a natural number of mutated codon in a particular genetic disease.

Step 2: Preparation of Probes

The probes are prepared such that the interrogated mutated codon is located at the center-most position of the oligonucleotide probe consisting of 7 nucleotides or more, rest of sequences are remained same as those of normal individuals and amine group is linked to 3′ terminus of the probe: wherein, one particular codon among the above-selected N interrogated codon is located at the center-most position of the oligonucleotide probe consisting of 7 nucleotides or more, and rest of sequences are remained same as those of normal individuals, i.e., one set of 4 probes are designed in a way that each probe has A, G, T, or C at the position of first nucleotide of the said interrogated codon and rest 2 nucleotides of the codon are remained same as those of normal individuals, the other set of 4 probes are designed in a way that each probe has A, G, T, or C at the position of second nucleotide of the said interrogated codon and rest 2 nucleotides of the codon are remained same as those of normal individuals, and another set of 4 probes are designed in a way that each probe has A, G, T, or C at the position of third nucleotide of the said interrogated codon and rest 2 nucleotides of the codon are remained same as those of normal individuals, finally to give 12 probes for interrogated mutated codon.

By using the above strategy, it is possible to manufacture the DNA chip which can detect and identify mutations with accuracy. It will be understood by the skilled in the art that the best discrimination for hybridization specificity can be attained by using oligonucleotide probes comprising 7 nucleotides or more, and locating the interrogated target nucleotide at the center-most position.

Furthermore, by using the codon scanning algorithm of the invention, DNA probes for multiplicity of genetic diseases can be designed, to explain it concretely, a total of 12N·D probes can be designed by repeating above steps for multiplicity (D) of genetic diseases.

The process for preparing DNA chip of the invention comprises a step of immobilizing the probes prepared above on the solid surface by the aid of spotting technique: the amine-linked probes prepared above are dissolved in a buffer solution of 1 to 7×, preferably 2 to 5×, more preferably 3×SSC (0.45M NaCl, 15 mM C₆H₅Na₃O₇, pH 7.0), and then spotted onto the aldehyde-derivatized solid surface using a microarrayer followed by immobilization of the probes on the solid surface via amine-aldehyde reaction. Herein, the solid materials to be used for immobilization of probes include, but not limited to, preferably glass plate, the concentration of probe is preferably 10 pmol/μl or higher, more preferably 50 pmol/μl or higher, most preferably 100 pmol/μl or higher, and binding reaction of amine group in probe to aldehyde-derivatized solid surface is performed under a condition of 70 to 90% humidity, preferably 80%, for 4 to 8 hours, preferably 5 to 7 hours, most preferably 6 hours.

The method for detecting genetic mutations employing the DNA chip of the invention comprises steps of: performing PCR using DNA to be interrogated and primers labeled with fluorescent material to obtain sample DNA labelled with fluorescent material; binding the sample DNA to the DNA chip at 10 to 37° C. for 3 to 13 hours, followed by washing the DNA chip; and, measuring fluorescent signal remained on the washed DNA chip. Herein, binding of sample DNA to the DNA chip is carried out under a condition of 3 to 10× binding buffer (SSPE: 0.15M NaCl, 10 mM NaH₂PO₄ H₂O, 1 mM EDTA, pH 7.4), at 10 to 37° C., preferably 20 to 30° C., most preferably 35° C., for 6 to 10 hours, most preferably 8 hours or longer. And, the DNA chip is preferably washed with first washing solution (3×SSPE) for 5 min and second washing solution (2×SSPE) for 5 min in a sequential order.

Since the DNA chip of the invention can be manufactured to identify over 10,000 selected mutations in parallel without failure by increasing integration of DNA chip, it can be applied to diagnose all kinds of genetic mutation-associated diseases, as well as identify mutations such as SNP. Furthermore, errors made in interpretation of results due to base pair mismatches found with DNA probes designed by conventional algorithms can be avoided by analysing the region surrounding the interrogated codon. Moreover, only mutation signal is detected for the homozygous mutations, and normal and mutation signals are simultaneously detected for the heterozygous mutations, making it possible to detect and identify mutations accurately such as discerning homozygous mutations from heterozygous mutations.

As an example of the invention, a DNA chip for diagnosis of Wilson disease which is a genetic disease was manufactured in accordance with the steps described above: the DNA probe was prepared to interrogate the genetic codon encoding mutated amino acids found in Wilson disease patients. Wilson disease, one of well-known genetic disorders, is caused by mutations in the gene for copper transporting protein and characterized by build-up of intracellular hepatic copper with subsequent hepatic and neurologic abnormalities. Until now, diagnosis of genetic disorders including Wilson disease has been carried out by amplifying the gene using each exon specific PCR marker (see: Tumer, Z., et al, Am. J. Hum. Genet., 60:63-71, 1997) followed by analysis of nucleotide sequence to compare with the published mutation databases, which essentially accompanies very time-consuming and costly step of PCRs for preparing exon DNA and for its sequencing, and identification of every mutation from entire sequence.

The present inventors prepared DNA chip for diagnosis of Wilson disease based on the codon scanning algorithm, and examined whether Wilson disease can be diagnosed using the DNA chip with ease, simplicity, accuracy and low cost compared to the conventional diagnosis methods which are complicated and expensive: First, genetic codons encoding changed amino acids found in Wilson disease patients were selected as mutated codons to be interrogated, and DNA probes were prepared based on the codon scanning algorithm of the invention. The probes were immobilized on the glass plate via spotting and binding reaction between amine and aldehyde to manufacture the DNA chip for diagnosis of Wilson disease. Then, DNA of interrogated region was prepared by using multiplex PCR. And then, based on the guideline for the analysis of the results obtained with DNA from normal individual, DNA from patient was successfully identified. Further, the inventors prepared DNA chip which can identify 16 mutations including 2 additional mutations found in Korean patients by supplementing 2 more probes to the DNA chip manufactured earlier, and found that it can be successfully applied for the identification of genetic mutation of Wilson disease, demonstrating that the DNA chip can be used for diagnosis of various genetic disorders.

Therefore, it can be speculated that the process for preparing DNA chip of the invention and probe immobilization technique via spotting can be applied to the diagnosis of various genetic mutation-associated disease and the diagnosis of multiplicity of genetic diseases in parallel as well as the identification of Wilson disease related mutations.

The present invention is further illustrated in the following examples, which should not be taken to limit the scope of the invention. Accordingly, it is understood by the conventionally skilled in the art that DNA chips for diagnosis of genetic diseases prepared by using codon scanning algorithm are fallen within the scope of the present invention.

### EXAMPLE 1

### Selection of the Mutations to be Interrogated

In order to manufacture DNA chip for diagnosis of mutations causing Wilson disease, the mutations to be interrogated were selected. For successful diagnosis of Wilson disease, the informations about nucleotide and amino acid sequence of ATP7B protein which is the cause of Wilson disease, were obtained from NCBI (National Center for Biotechnology Information)-affiliated gene databases, GenBank and OMIN (Online Medelian Inheritance in Man), as well as informations on the allelic variants of the disease were obtained. Furthermore, based on above-obtained amino acid sequence information and the report obtained from a published literature (see: Kim, E. K., Ph.D. thesis, KAIST, 1999), ATP7B protein was examined for functional motifs. A functional motif of a protein is a short stretch of amino acid sequence representing a particular function of the protein, and following functional motifs have been found in the ATP7B protein; a copper-binding motif GMTCXXC at the N-terminal side of the protein, a transduction domain which has homology to TGES/A amino acid sequence, a cation channel motif CPC, a phosphate domain DKTGT, an ATP bindig domain TGDN at the C-terminal side of the protein, and a hinge region MXGDXNDX, wherein X refers to any amino acid not a particular amino acid. According to HGMD (Human Gene Mutation Database), Wilson disease has been known to be caused by 12 types of variant ATP7B protein encoded by 12 types of gene with substitution mutation in functionally important domains including phosphate domain, ATP binding domain and hinge domain, and among the mutations found in 12 types of protein, Asn1270 mutation has been reported to be found in Korean Wilson disease patients. Therefore, the present inventors have selected, as mutations to be interrogated, 14 substitution mutations leading 14 variant proteins including Arg778Leu (see: Kim, E. K., et al, Hum. Mutat., 11:275-278, 1998) which represented 37.5% of Wilson disease alleles in Korean patients and His1069Gln(see: Payne, A., et al, Proc. Natl. Acad. Sci., USA, 95:10854-10859, 1998) which found in patients in western population with considerable frequency.

### EXAMPLE 2

### Amplification of the Interrogated Region by PCR

To analyze 14 mutations mentioned above, 4 pairs of PCR primer were prepared, respectively. For amplification of 778Arg region (91 bp) frequently found in the Korean patients, primer 1: 5′-GCCCTGTGACATTCTTCGA-3′ (SEQ ID NO: 1) and primer 2: 5′-GCTGCTGTTACCTTTGCC A-3′ (SEQ ID NO: 2) were designed. Fluorescein phosphoamidite (F, Molecular Dynamics, CA, USA) was linked to the hydroxyl group at the 5′-terminus of the strand complementary to the probe. For amplification of the region of 1069His frequently found in western patients and the phosphate domain, both are located in the same exon (173 bp), primer 3: 5′-GATGTTTGACAAGACTGGCA-3′ (SEQ ID NO: 3) and primer 4: 5′-CCTCTTTACAGTATTTGGTGA-3′ (SEQ ID NO: 4) were used. PCRs were peformed for ATP binding domain (128 bp) using primer 5: 5′-CAATCGCAGACGCTGTCAA-3′ (SEQ ID NO: 5) and primer 6: 5′-CTGTACCTGGGTGGCAATA-3′ (SEQ ID NO: 6), and for hinge region using primer 7: 5′-TAAAGGGAAGAAAGTCGCCA-3′ (SEQ ID NO: 7) and primer 8: 5′-GCTGCCTCGATGGCCACA-3′ (SEQ ID NO: 8). PCRs were performed using over 100 ng DNA obtained from the sample blood as templates in a total volume of 50 μl under a following condition: one cycle of denaturation at 96° C. for 8 min; 30 cycles of denaturation at 92° C. for 1 min, annealing at 57° C. for 1 min, and extension at 72° C. for 30 sec; plus one cycle of extension at 72° C. for 7 min.

To avoid primer dimerization, 3′-termini of primers to which DNA polymerase binds were designed to be adenine uniformly if possible, and all primers were designed to anneal around 58° C., making it possible to amplify all 4 templates simultaneously.

### EXAMPLE 3

### Preparation of Probes for Identification of Mutations

To identify 14 substitution mutations above, the probes were prepared using the codon scanning algorithm described above: i.e., to analyze nucleotide sequence variations leading to one amino acid substitution, one codon comprising 3 nucleotides specifying one amino acid was selected as a codon to be interrogated. And, to interrogate each selected codon, one set of four 15mer-probes were designed per one nucleotide of a codon in a way that the 8th position which is the center-most position of the 15mer-probe was substituted with A, G, T or C while the other sequences in the probe remain unchanged, finally to give 12 probes for analysis of one amino acid mutation.

The probes were designed such that probe N-1(1st nucleotide of the wildtype codon), probe N-5(2nd nucleotide of the wildtype codon) and probe N-9(3rd nucleotide of the wildtype codon), wherein N refers to the amino acid number to be interrogated (natural number from 1 to 14), designate the reference (control) probes which have nucleotide sequence complementary to wildtype codon, and this strategy was applied to all amino acids to be interrogated. For example, in case of Arg778 (designated as amino acid No. 1), probes 1-1(C was located at 8th position which is the center-most position of the probe), 1-5(G), and 1-9(G) were designed to detect nucleotide sequence of normal codon, while, Arg778Leu mutation would not give any signal with above reference probes but hybridize only to the probe 1-8 which has complementary sequence to CTG.

By same procedure described above, probes were designed for His1069(amino acid No. 2), Gly1035(amino acid No. 3), Arg1038(amino acid No. 4), Arg1041(amino acid No. 5), Gly1213(amino acid No. 6), Val1216(amino acid No. 7), Thr1220(amino acid No. 8), Asp1222(amino acid No. 9), Gly1266(amino acid No. 10), Asp1267(amino acid No. 11), Asn1270(amino acid No. 12), Pro1273(amino acid No. 13) and Ala1278(amino acid No. 14), and among the probes to interrogate each amino acid described above, 2-10 (His1069Gln), 3-8(Gly1035Val), 4-6(Arg1038Lys), 5-4(Arg1041Trp), 6-8 (Gly1213Val), 7-2(Val1216Met), 8-8(Thr1220Met), 9-4(Asp1222Tyr), 10-2(Gly1266Arg), 10-8(Gly1266Val), 11-6(Asp1267Ala), 12-7(Asn1270Ser), 13-8(Pro1273Leu) and 14-8(Ala1278Val) were the probes for detecting mutations. During the synthesis of above probes using Aminolinker column (Cruachem, Glasgow, Scotland), nucleotide with amine residue was used as the first nucleotide at 3′-terminus.

### EXAMPLE 4

### Immobilization of DNA Probes

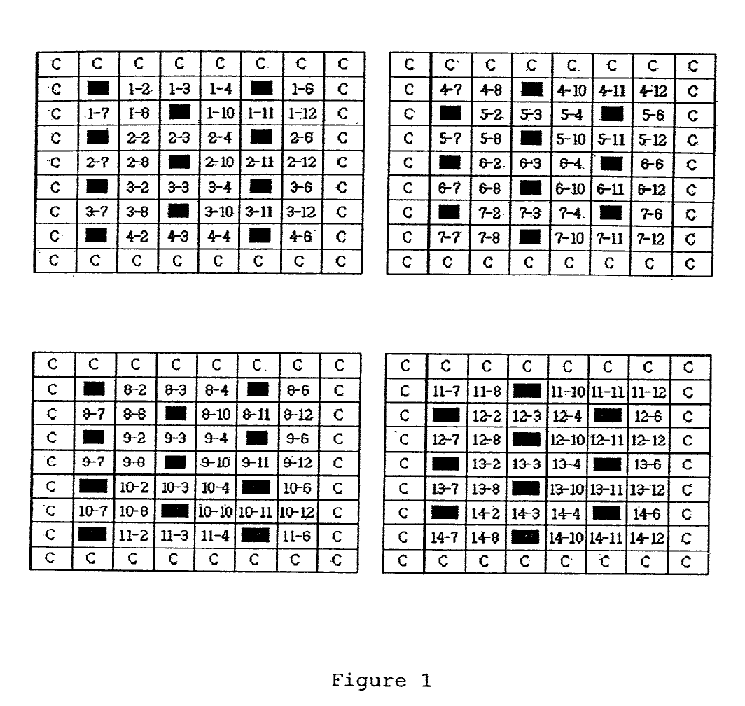

To immobilize the probes prepared in Example 3 onto the glass plate, probes were spotted (see: Yoon, S. H., et al, J. Microbiol. Biotechnol., 10(1): 21-26, 2000) onto the glass plate derivatized with aldehyde (CEL Associates, Inc., Houston, Tex., USA) using a microarrayer under a buffer condition of 3×SSC (0.45M NaCl, 15 mM C₆H₅Na₃O₇, pH 7.0), and then amine-aldehyde binding reaction was performed for 2 hours under a condition of over 80% humidity, followed by incubation for 6 hours. Here, the concentration of reference probe (control) was fixed at 10 μM, and concentration of the probes for detecting mutations was fixed at 100 μM (see: FIG. 1). To measure the signal with ease, 5′ terminus of each probe was labeled with fluorescein phosphoamidite. FIG. 1 is a schematic representation of the DNA chip immobilized with 14 sets of 12 probes (12 probes per one codon) for the said 14 codons to be interrogated, in which a total of 288 probes comprising 168 probes for mutation sequences and 120 probes for reference sequences were packed in order at the predetermined positions on an area of 0.8 cm². In this illustration, dark region represent probes for normal nucleotide sequence and C represent control probe.

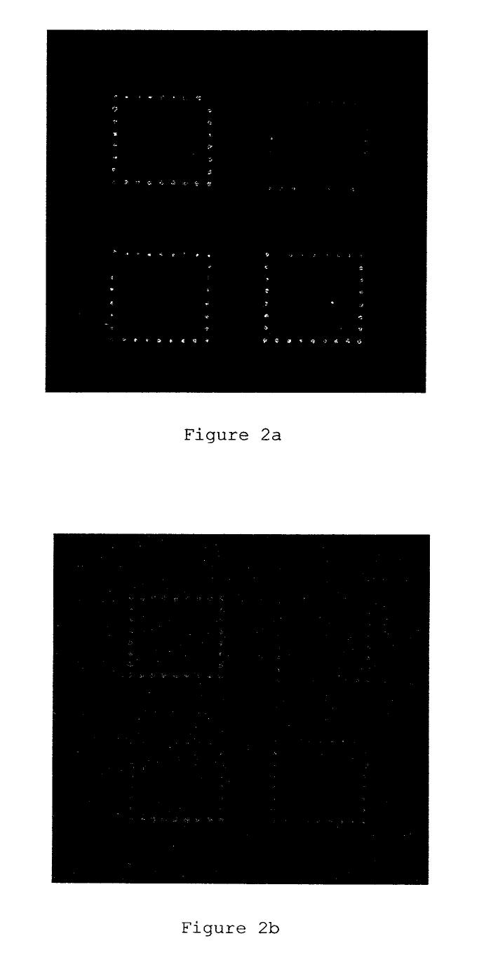

To assess the degree of immobilization of probes onto glass plates, hybridization and washing were performed. The chip was incubated in 10 μl of 6× binding buffer (SSPE: 0.15M NaCl, 10 mM NaH₂PO₄.H₂O, 1 mM EDTA, pH 7.4) omitting fluorescence-labeled DNA at 37° C. for 12 hours, and then, washed with 3×SSPE for 5 min, 2×SSPE for 5 min and 1×SSPE for 5 min in order, followed by detection of remained probes using ScanArray5000 (GSI Lumonics Inc., Bedford, Mass., USA) (see: FIGS. 2a and 2b). FIG. 2a is a photograph showing the DNA chip before treatments of binding and washing, and FIG. 2b is a photograph showing the DNA chip after treatments described above, and it has been found that there was sufficient amount of probes remained on the DNA chip to give meaningful results after binding and washing judged by control probe remained.

### EXAMPLE 5

### Evaluation of Functionality of DNA Chip using Reporter DNA Fragment

To evaluate functionality of DNA chip manufactured above and to optimize the condition of hybridization, reporter DNA fragments which can bind complementary to the immobilized probes were prepared. The Arg778Leu mutation which is found frequently in Korean Wilson disease patients was selected to be included in the mutant reporter DNA fragment, and then, a reporter of normal individual and a reporter of patient with mutation were prepared, respectively. The nucleotide sequence of reporter of normal individual is 5′-CAGCCACCGGCCCAGG-3′ (SEQ ID NO: 9) and 5′-CCAGCCACAGGCCCAGG-3′ (SEQ ID NO: 10) is for Wilson disease patient, where 5′-termini of all reporters were labeled with fluorescein phosphoamidite to give fluorescent signals.

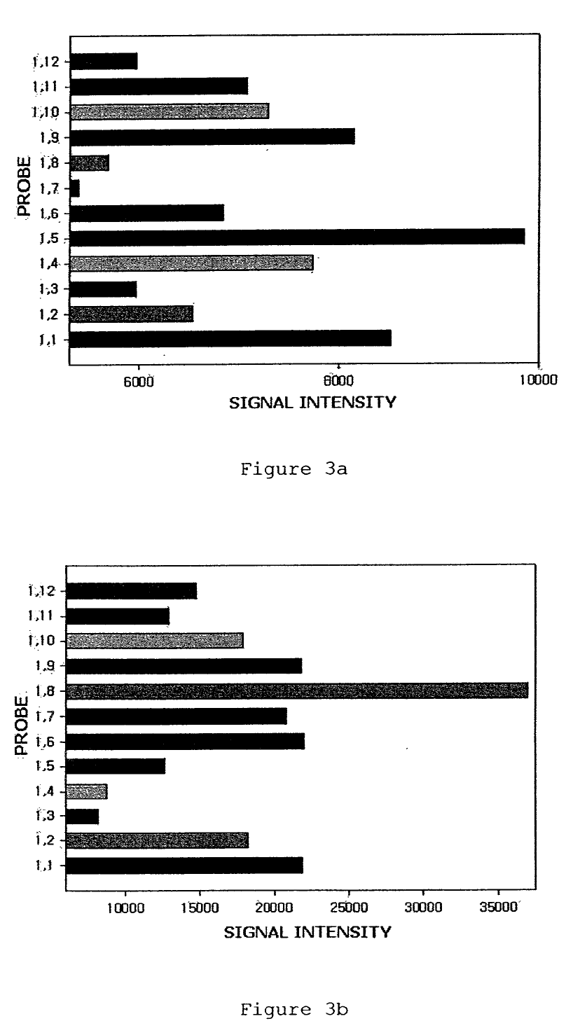

Ten microliter aliquots of 3×, 4×, 5×, 6×, and 7×SSPE containing 0.1 μM of each reporter DNA fragment prepared above were incubated with the DNA chip manufactured above for 4 hours at a room temperature, 30 and 37° C., respectively, to allow complementary binding, which was followed by washing with 3×SSPE for 5 min, 2×SSPE for 5 min, and 1×SSPE for 5 min and measuring intensities of signals with ScanArray5000 (see: FIGS. 3a and 3b). FIG. 3a is a graph showing the analysis of mutation of normal individual, and FIG. 3b is a graph showing the analysis of mutation of Wilson disease patients. As shown in FIG. 3a, probes 1-1, 1-5 and 1-9, which represent normal codon, gave hybridization signals as expected, and also these three probes showed stronger signal intensity compare to other probes when the signal intensity was analyzed using QuantArray (GSI Lumonics Inc., Bedford, Mass., USA). As shown in FIG. 3b, mutant reporter produce signal only with probe 1-8 which represent mutated codon. From these results, it has been found that the strongest signal intensity can be obtained under a hybridization condition of 6×SSPE and 0° C.

### EXAMPLE 6

### Analysis of DNA from Normal Individual Employing Single Strand PCR and Establishment of Guideline for Result Analysis

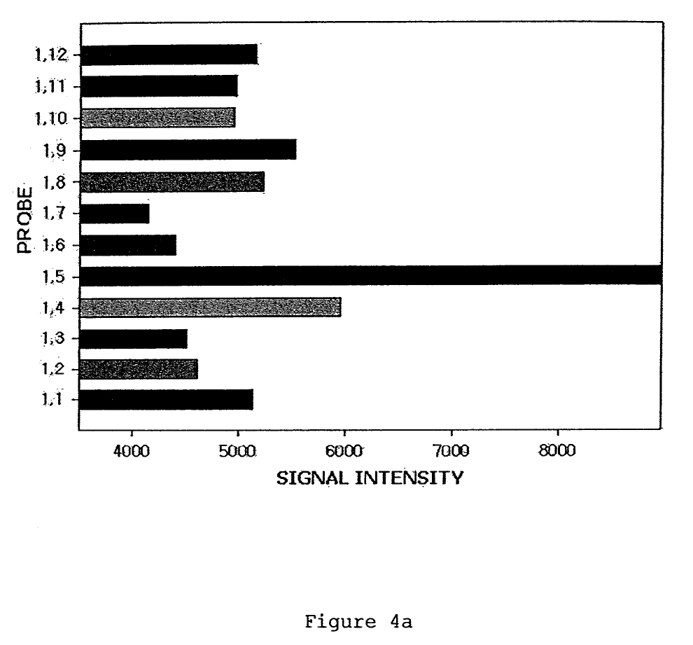

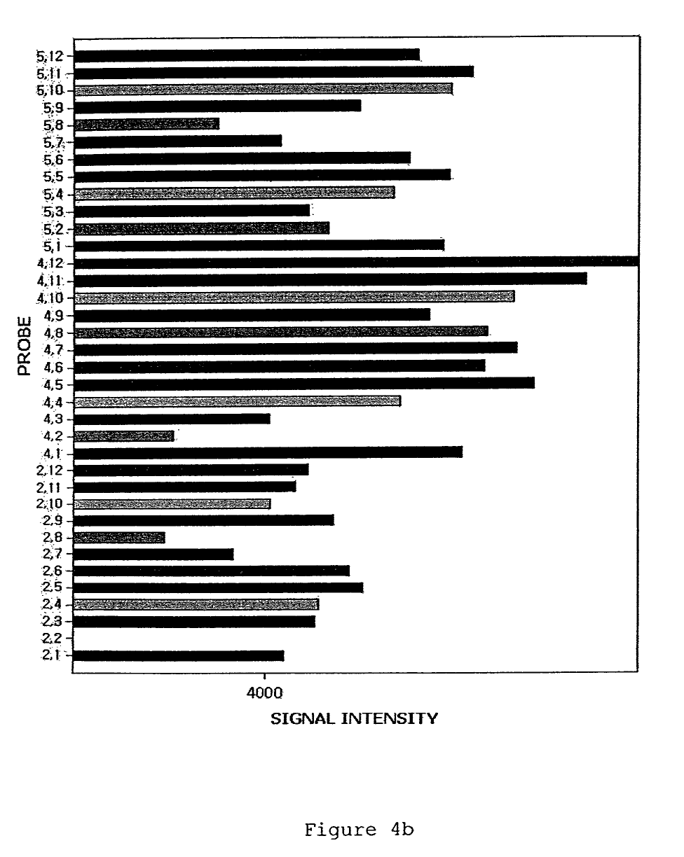

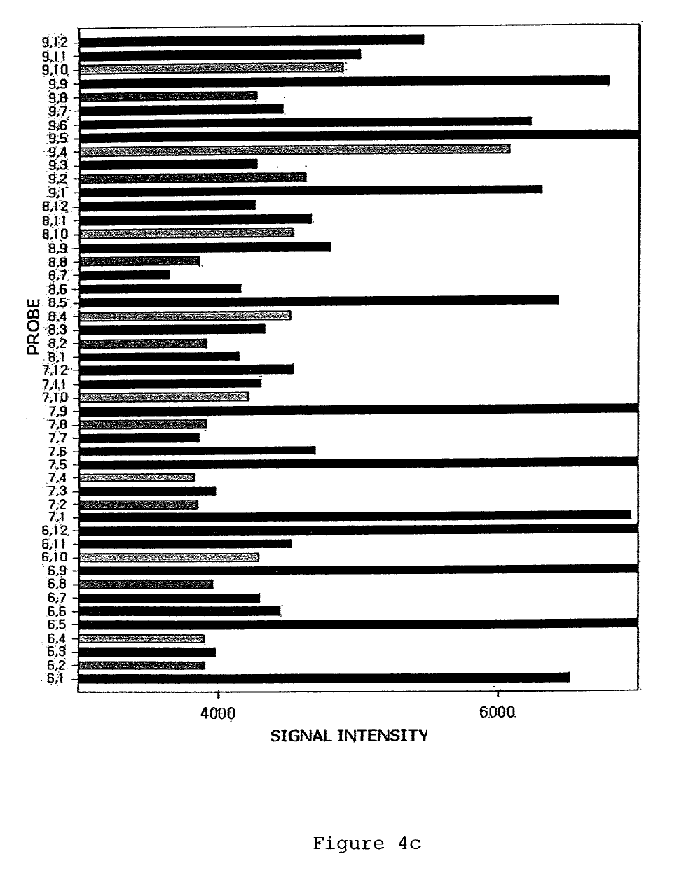

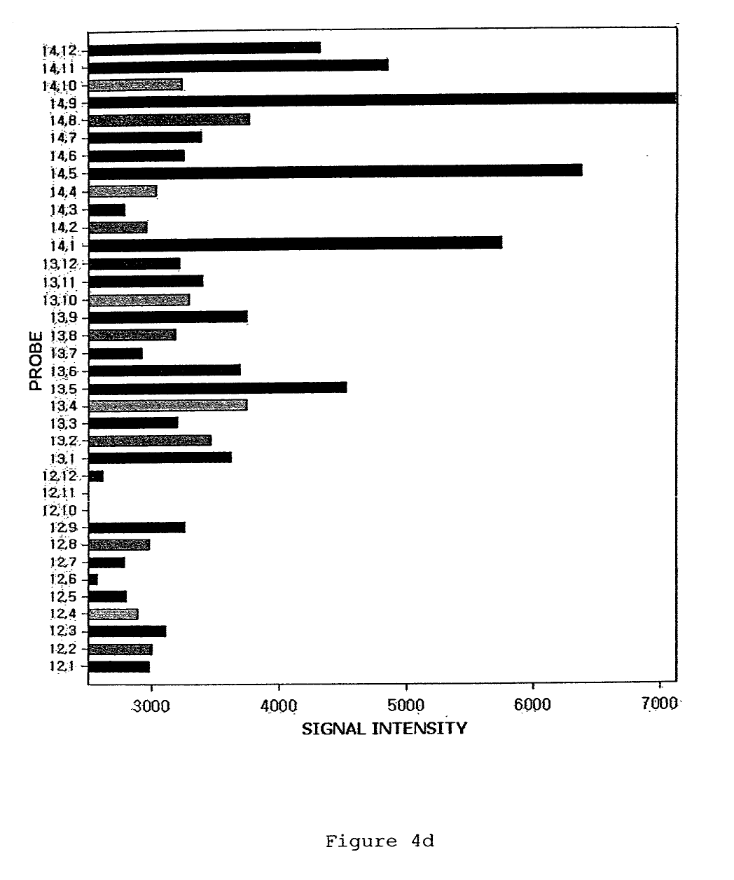

Fourteen interrogated regions of normal individual were amplified respectively by single strand PCR and then analyzed for mutation using the DNA chip manufactured above. The DNA fragment of interrogated region was amplified by PCR in the presence of both primers, and then the PCR product was used as template for another PCR using one primer with fluorescence label to obtain fluorescence-labeled DNA. The DNA chip of the invention was incubated with 10 μl 6×SSPE containing 1 ul of single strand PCR product obtained above at 30° C. for 4 hours to induce hybridization, and then washed with 3×SSPE for 5 min and 2×SSPE for 5 min. The signals were measured using ScanArray5000 (see: FIGS. 4a, 4b, 4c and 4d). FIG. 4a is a graph showing the analysis of mutation frequently found in Korean Wilson disease patients (probe 1) using DNA chip of the invention, FIG. 4b is a graph showing the analysis of mutation frequently found in Wilson disease patients in western population and in phosphate domain using DNA chip of the invention (probes 2 to 5), FIG. 4c is a graph showing the analysis of mutation found in ATP binding domain using DNA chip of the invention (probes 6 to 9), and FIG. 4d is a graph showing the analysis of mutation found in hinge domain using DNA chip of the invention (probes 10 to 14). As shown in FIGS. 4a to 4d, after induction of complementary binding of each interrogated region to various probes, DNA from normal individual showed positive signals with 778Arg(probe 1), 1069His(probe 2), 1038Arg(probe 4), 1041Arg(probe 5), 1213Gly(probe 6), 1216Val(probe 7), 1220Thr(probe 8), 1222Asp(probe 9), 1270Asn(probe 12), 1273Pro(probe 13) and 1278Ala(probe 14), indicating that the interrogated DNA was normal.

Based on the results above, the case showing the strongest signal in two or more probes containing normal amino acid codon was determined to be ‘normal’, and the case showing the strongest signal in the probe containing mutated codon among total probes (12 probes for one amino acid) was determined to be ‘mutation’, the case showing normal signal and mutation signal in parallel was determined to be ‘heterozygous mutation’, and the case showing no normal signal but mutation signal was determined to be ‘homozygous mutation’.

### EXAMPLE 7

### Analysis of DNA from Wilson Disease Patient Employing Single Strand PCR

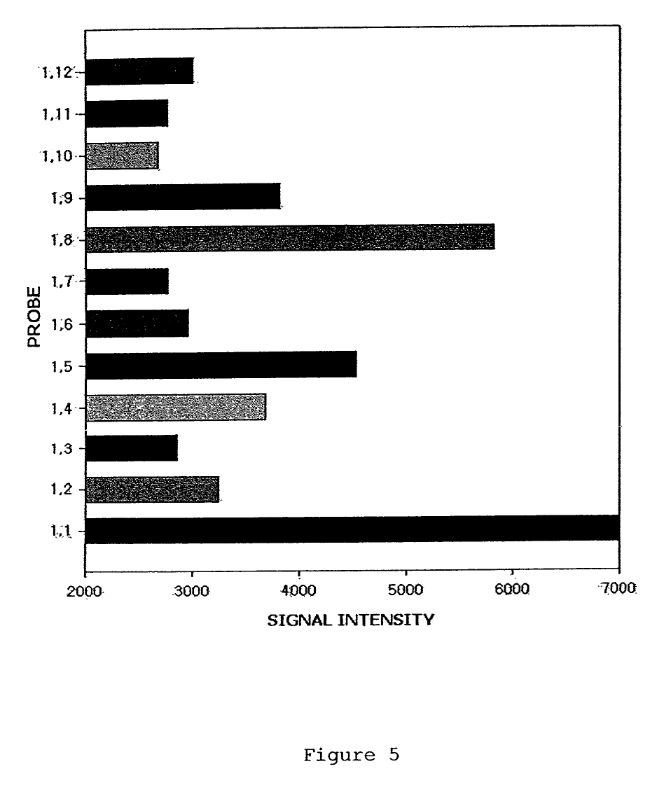

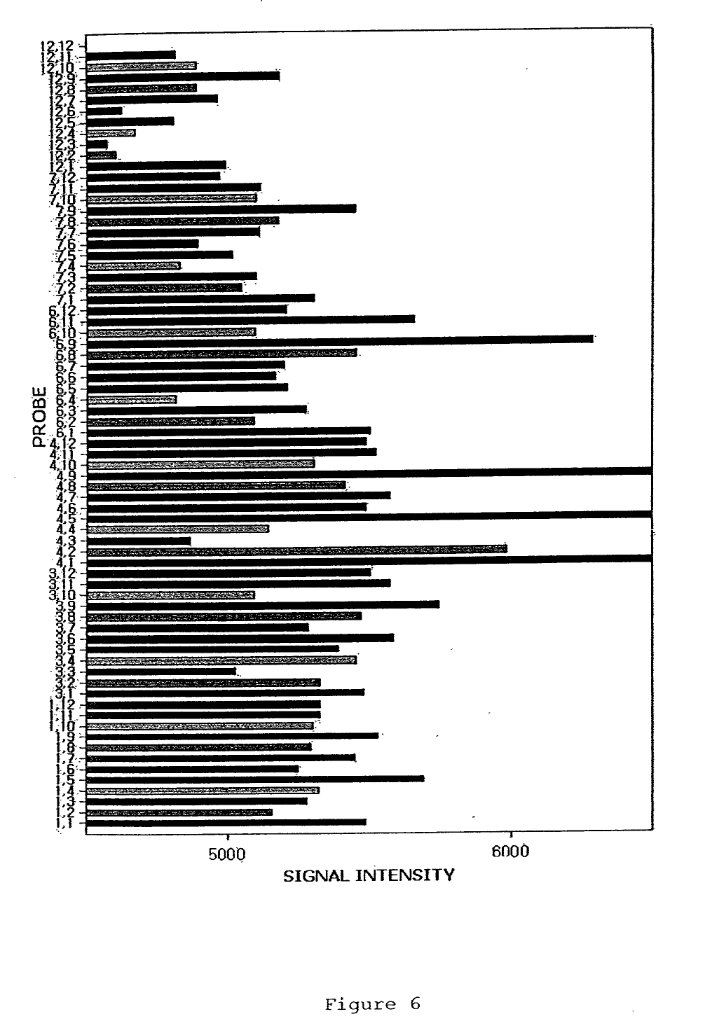

Fourteen interrogated regions of DNA from Wilson disease patient with Arg778Leu were amplified by single strand PCR and then analyzed using the DNA chip of the invention (see: FIG. 5). FIG. 5 is a graph showing the analysis of Arg778Leu(probe 1) mutation in the patient DNA. After induction of complementary binding of each interrogated region to various probes, substitution mutation was detected in Arg778Leu frequently found in the Korean patient, and the mutation was found to be heterozygous mutation in which one chromosome carries normal sequence and the other carries mutated sequence. In the analysis carried out based on the presumption that other interrogated regions of this patient DNA except Arg778Leu were normal, 1069His(probe 2), 1038Arg(probe 4), 1041Arg(probe 5), 1213Gly(probe 6), 1216Val(probe 7), 1220Thr(probe 8), 1222Asp(probe 9), 1270Asn(probe 12) and 1273Pro(probe 13), 1278Ala(probe 14) showed the result of ‘normal’.

Therefore, the DNA chip manufactured by the said process can be applied for detection of mutations in the DNA from Wilson disease patients.

### EXAMPLE 8

### Analysis of DNA from Normal Individual Employing Multiplex PCR

Fourteen interrogated regions of DNA from normal individual were amplified by multiplex PCR described in Example 2, and then, analyzed using the DNA chip of the invention. That is, 10 ul of 6×SSPE containing 1 ul of multiplex PCR product was prepared, heat-treated at 98 C for 5 min to make DNA single-stranded, cooled down on an ice bath for 1 min, and then annealed to DNA chip of the invention at 30 C for 8 hours or longer, which was followed by washing with 3×SSPE for 5 min, 2×SSPE for 5 min, and analysis using ScanArray5000 (see: FIG. 6). FIG. 6 is a graph showing the analysis of multiplex PCR amplified DNA from normal individual. Among 14 mutations, probes 1, 3, 4, 6, 7 and 12 showed the result of normal, thus, by using the condition determined in this Example, a large number of various interrogated regions can be analyzed in parallel with ease, as well as diagnosis of other genetic diseases than Wilson disease can be performed in parallel by using one DNA chip.

### EXAMPLE 9

### Manufacture of a DNA Chip for Diagnosis of Wilson Disease in Korean Population by Supplementing Probes

To diagnose Wilson disease more accurately and to find out the possibility of supplementing more probes to the DNA chip, which is the distinguished merit of the DNA chip of the invention, the probes for mutations Ala874Val (amino acid No. 15) and Leu1083Phe (amino acid No. 16) which are another mutations found in Wilson disease patients were prepared additionally. The said two probes were prepared by employing the same algorithm used for preparation of the probes earlier, and a new DNA chip detecting 16 mutations was manufactured in a way that mutation signals can be detected with probe 16-8 for Ala874Val mutation and probe 17-4 for Leu1083Phe mutation.

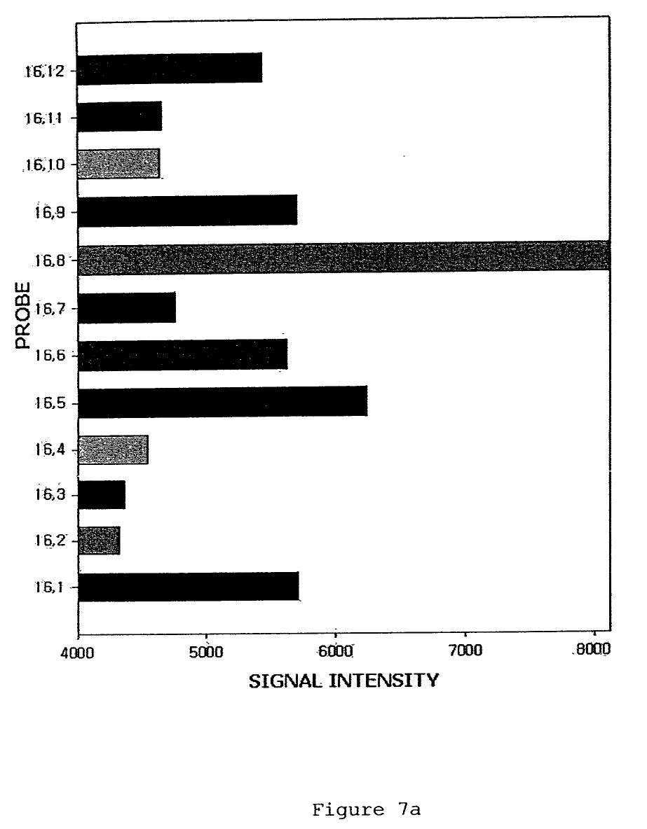

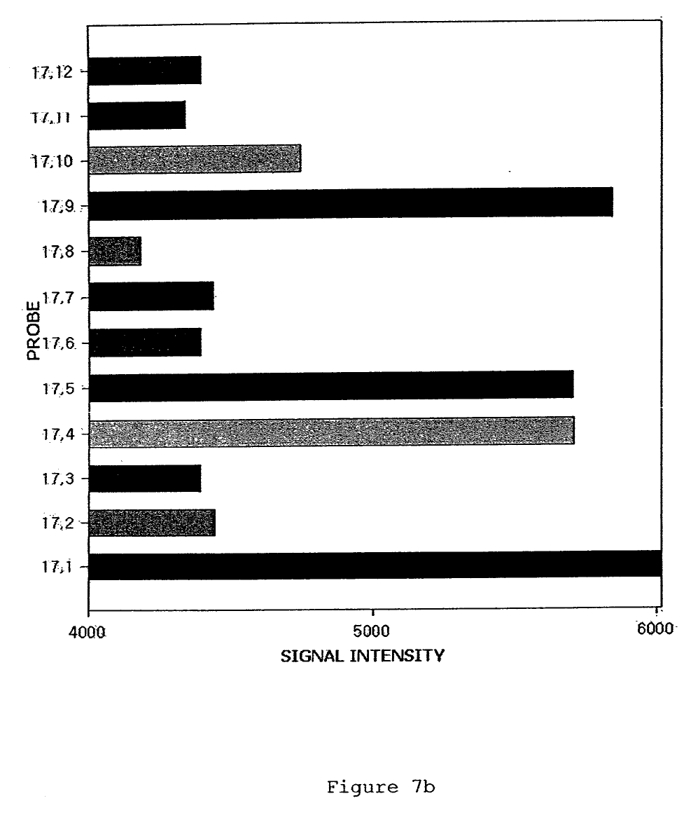

Additional primers were prepared to obtain two DNA fragments containing interrogated mutated regions. The region containing Ala874Val mutation was amplified by PCR using primer 9: 5′-CTACGTCTAGGAGAAGCCA-3′ (SEQ ID NO: 11) and primer 10: 5′-GAGCACAGAGCCATGTGCA-3′ (SEQ ID NO: 12), and the region containing Leu1083Phe mutation was amplified by PCR using primer 11: 5′-CTTTCACTTCACCCCTCT-3′ (SEQ ID NO: 13) and primer 12: 5′-TGCCTGGAAGTCCGTGCA-3′ (SEQ ID NO: 14). The PCR product was subjected to single strand PCR to prepare single-stranded DNA of which specific hybridization to the probe on the DNA chip was evaluated (see: FIGS. 7a and 7b). FIG. 7a is a graph showing the analysis of Ala874Val mutation in the DNA from a patient and FIG. 7b is a graph showing the analysis of Leu1083Phe mutation in the DNA from a patient using the DNA chip manufactured in this Example. As shown in FIG. 7a, Ala874Val mutation of the patient was detected to be a heterozygous mutation with success, and as shown in FIG. 7b, Leu1083Phe mutation of the other patient was also detected to be a heterozygous mutation with success.

As shown in the results above, more probes to interrogate the mutation can be added onto the DNA chip of the invention whenever additional mutations causing Wilson disease are uncovered, and it has been found that manufacturing of DNA chip for diagnosis of a multiplicity of genetic diseases in parallel became possible by adding the probes not only for Wilson disease but also for other genetic diseases onto the DNA chip of the invention.

### EXAMPLE 10

### Application of the DNA Chip to the Unknown Patients

Blood samples taken from two Koreans (patient A and patient B) who were not identified as normal or diseased individual were analyzed for genetic disease using the DNA chip of the invention. The DNA chip manufactured in Example 9 was employed to diagnose the unknown patients, which revealed that patient A had no mutation of all 16 amino acids, and patient B had heterozygous mutations of Arg778Leu and Ala874Val. To establish the reliability of the procedure above, the interrogated regions of DNA from the patients were subjected to a conventional nucleotide sequencing, from which the patient A was confirmed to be a normal individual and the patient B was confirmed to be a Wilson disease patient. Thus, it has been found that mutations in DNA from Wilson disease patient can be successfully identified by using the DNA chip of the invention.

As clearly illustrated and demonstrated above, the present invention provides a process for preparing oligonucleotide probes which are designed to detect mutations in the entire interrogated codon regions determined by codon scanning algorithm, a process for preparing DNA chip using the probes prepared by the said process, a DNA chip prepared by the said process and a method for detecting mutations using the said DNA chip. The process for preparing probes using the codon scanning algorithm comprises the steps of: selecting mutated codon to be interrogated; and, preparing the probes such that the interrogated mutated codon is located at the center-most position of the oligonucleotide probe consisting of 7 nucleotides or more, rest of sequences are remained same as those of normal individuals and amine group is linked to 3′ terminus of the probe. And, the process for preparing DNA chip comprises a step of immobilizing the probes prepared above on solid surface by spotting technique, and the method for detecting genetic mutations employing the said DNA chip comprises the steps of reacting fluorescence-labeled sample with the DNA chip, washing, and measurement of fluorescent signal remained on the washed DNA chip. By using the DNA chip of the invention, errors made in interpretation of results due to base pair mismatches found with DNA probes designed by conventional algorithms can be avoided, homozygous mutations can be discerned from heterozygous mutations, mutations causing various genetic diseases can be detected and identified in a rapid and accurate manner, and DNA chip using codon scanning algorithm can be applied for the diagnosis of all genetic mutation-associated diseases and the identification of mutations such as SNP.

What is claimed is:

1. A process for preparing oligonucleotide probe using codon scanning algorithm which comprises the steps of:
(i) selecting a mutated codon to be interrogated; and,
(ii) preparing a probe such that the interrogated mutated codon is located at the center-most position of the oligonucleotide probe consisting of 7 nucleotides or more, rest of sequences are remained same as those of normal individuals and amine group is linked to 3′ terminus of the probe.

2. The process for preparing oligonucleotide probe of claim 1, wherein one set of 4 probes are designed in a way that each probe has A, G, T, or C at the position of first nucleotide of the interrogated codon and rest 2 nucleotides of the codon are remained same as those of normal individuals, the other set of 4 probes are designed in a way that each probe has A, G, T, or C at the position of second nucleotide of the said interrogated codon and rest 2 nucleotides of the codon are remained same as those of normal individuals, and another set of 4 probes are designed in a way that each probe has A, G, T, or C at the position of third nucleotide of the said interrogated codon and rest 2 nucleotides of the codon are remained same as those of normal individuals, finally to give 12 probes for interrogated mutated codon.

3. A process for preparing DNA chip which comprises a step of spotting the probe prepared by the process of claim 1 onto aldehyde-coated solid surface to immobilize the probe on the solid surface.

4. The process for preparing DNA chip of claim 3, wherein the immobilization is performed by a binding reaction of amine group in probe and aldehyde coated on solid surface

5. The process for preparing DNA chip of claim 4, wherein the binding reaction is performed under a condition of 70 to 90% humidity for 4 to 8 hours.

6. The process for preparing DNA chip of claim 3, wherein the solid material is a glass plate.

7. A DNA chip prepared by the process of claim 3.

8. A method for detecting genetic mutations using the DNA chip of claim 7 which comprises the steps of:
(i) performing PCR using DNA to be interrogated and primers labeled with fluorescent material to obtain sample DNA labelled with fluorescent material;
(ii) binding the sample DNA to the DNA chip at 10 to 37° C. for 3 to 13 hours, followed by washing the DNA chip; and,
(iii) measuring fluorescent signal remained on the washed DNA chip.

9. The method for detecting mutations using the DNA chip of claim 8, wherein the binding of sample DNA to DNA chip is carried out under a condition of 3 to 10× binding buffer (SSPE: 0.15M NaCl, 10 mM NaH₂PO₄.H₂O, 1 mM EDTA, pH 7.4).

10. The method for detecting mutations using the DNA chip of claim 8, wherein the the DNA chip is washed with first washing solution (0.45M NaCl, 30 mM NaH₂PO₄.H₂O, 3 mM EDTA, pH 7.4) for 5 min and second washing solution (0.3M NaCl, 20 mM NaH₂.PO₄ H₂O, 2 mM EDTA, pH 7.4) for 5 min in a sequential order.


SEQUENCE LISTING (14 sequences)

SEQ ID NO: 1 - DNA, 19 bp, Artificial Sequence
  primer 1
gccctgtgacattcttcga

SEQ ID NO: 2 - DNA, 19 bp, Artificial Sequence
  primer 2
gctgctgttacctttgcca

SEQ ID NO: 3 - DNA, 20 bp, Artificial Sequence
  primer 3
gatgtttgacaagactggca

SEQ ID NO: 4 - DNA, 21 bp, Artificial Sequence
  primer 4
cctctttacagtatttggtga

SEQ ID NO: 5 - DNA, 19 bp, Artificial Sequence
  primer 5
caatcgcagacgctgtcaa

SEQ ID NO: 6 - DNA, 19 bp, Artificial Sequence
  primer 6
ctgtacctgggtggcaata

SEQ ID NO: 7 - DNA, 20 bp, Artificial Sequence
  primer 7
taaagggaagaaagtcgcca

SEQ ID NO: 8 - DNA, 18 bp, Artificial Sequence
  primer 8
gctgcctcgatggccaca

SEQ ID NO: 9 - DNA, 16 bp, Artificial Sequence
  reporter for normal individual
cagccaccggcccagg

SEQ ID NO: 10 - DNA, 17 bp, Artificial Sequence
  reporter for Wilson′s disease patient
ccagccacaggcccagg

SEQ ID NO: 11 - DNA, 19 bp, Artificial Sequence
  primer 9
ctacgtctaggagaagcca

SEQ ID NO: 12 - DNA, 19 bp, Artificial Sequence
  primer 10
gagcacagagccatgtgca

SEQ ID NO: 13 - DNA, 18 bp, Artificial Sequence
  primer 11
ctttcacttcacccctct

SEQ ID NO: 14 - DNA, 18 bp, Artificial Sequence
  primer 12
tgcctggaagtccgtgca